(19) United States
(12) Patent Application Publication
(10) Pub. No.: US 2002/0012938 A1
(43) Pub. Date: Jan. 31, 2002

(54) Detection of epithelial dysplasia

(76) Inventors: Mark Rutenberg, Suffern, NY (US); Drore Eisen, Cincinnati, OH (US); Stephen Frist, Monsey, NY (US); Donald Alan Kristt, Petach Tikvah (IL)

Correspondence Address: Peter L. Berger, Esq., LEVISOHN, LERNER, BERGER & LANGSAM, Suite 2400, 757 Third Avenue, New York, NY, 10017, US

(21) Appl. No.: 09/922,013

(22) Filed: Aug. 3, 2001

Related U.S. Application Data
(63) Continuation-in-part of application No. 09/298,218, filed on Apr. 23, 1999, now Pat. No. 6,284,482.
(63) Continuation-in-part of application No. 09/298,219, filed on Apr. 23, 1999, now Pat. No. 6,297,044.
(60) Provisional application No. 60/121,255, filed on Feb. 23, 1999.

(51) Int. Cl. C12Q 1/68; G01N 33/567; G06F 19/00; G01N 33/48; G01N 33/50

(52) U.S. Cl. 435/6; 435/7.21; 702/19; 702/20; 435/40.5

(57) ABSTRACT

A system to detect epithelial dysplasia is disclosed by examining cells obtained by a non-lacerational trans-epithelial device. The system selects suspect cells based on abnormal morphology, cytometry and/or using molecular diagnostic techniques. In a preferred embodiment, the results of the computer analysis are displayed as a histogram and the images of the suspect cells are reviewed for DNA ploidy by a pathologist on a cell by cell basis.

### RELATED APPLICATIONS

The present application relates to and is a continuation-in-part of U.S. Nonprovisional Application Ser. No. 09/298,218 filed Apr. 23, 1999 (“the '218 application”); U.S. Nonprovisional Application Ser. No. 09/298,219 filed Apr. 23, 1999 (“the '219 application”); and U.S. Provisional Application Ser. No. 60/225,186 filed Aug. 14, 2000 (“the '186 application”). The disclosures of those applications are fully incorporated herein by reference.

### FIELD OF THE INVENTION

The present invention relates to a method for the detection of epithelial dysplasia using molecular diagnostic techniques either independently or in conjunction with a DNA ploidy analysis.

### BACKGROUND OF THE INVENTION

The most common cancer of the oral mucosa is squamous cell carcinoma. Pre-malignant lesions have been traditionally identified based on light microscopic, histological criteria for epithelial dysplasia. However, the microscopic assessment of epithelial dysplasia is very subjective, and consequently often unreliable. Accurate, reproducible agreement among experienced board-certified oral pathologists diagnosing oral epithelial dysplasia is difficult to achieve, according to the study by Abbey, et al. In fact, these workers showed that a given pathologist agrees exactly with his own microscopic diagnoses of epithelial dysplasia in only 50.8% of cases.

Additionally a histological diagnosis of epithelial dysplasia, per se, does not ensure that a pre-malignant lesion will undergo malignant transformation some time in the future. Actually, many dysplastic lesions never transform into carcinoma. Further complicating the prognostic assessment of oral mucosal epithelial changes is the observation that some oral lesions transform, de novo, into carcinoma without passing through a dysplastic, pre-malignant stage.

For these reasons, new diagnostic approaches need to be utilized. The goal is to more reliably identify the earliest oral epethelial changes that destine a cell to progress towards cancer. Since the ultimate pathogenetic basis for cancer lies in damage to the gene-level growth control mechanisms of a cell, molecular biological approaches would seem a natural direction for improved diagnosis of pre-malignant status. A number of workers have begun to apply molecular biological methods to the oral mucosa in an effort to delineate specific genomic alterations characteristic of the progressive stages in the development of oral cancer. Detection of genetic changes in the oral mucosa has been extensively studied as a method of identifying oral lesions that are dysplastic and cancerous as well as lesions with potential to transform into cancerous lesions. For instance, the fluorescence in situ hybridization (FISH) technique has been shown to be effective in demonstrating chromosomal aberrations in head and neck cancers that are likely to exist prior to the appearance of histological changes. Gene level amplification of the growth and cell-cycle regulator, cyclin D1, also has been demonstrated in cancers of the head and neck.

Other genetic abnormalities observed in Squamous Cell Carcinomas (SCC) of this region include the loss of heterozygosity (LOH) at chromosomal sites on 3p, 9p, 11p, 11q and 17p. The loss of heterozygosity on 3p, 9p, and 17p in pre-malignant lesions as well, indicates that these alterations may play an important role in the earliest stages of transformation. It is in these initial phases of neoplastic change that such molecular biological information would provide a critical adjunct to the histopathological findings. Similarly, superficial exfoliative cell samples have been used to map clonal genetic alterations in the oral epithelium. In this way, allelic gene loss has been detected not only in oral cancers, but in precancers as well.

Pre-malignant lesions and carcinomas have also been investigated immunocytochemically for the expression of the protein product of the p53 tumor suppressor gene. Although p53 is an early event in oral carcinogenesis, immunohistochemistry cannot always detect changes in p53 expression in oral precancerous lesions. Another immunocytochemically-detected protein, CD44 variant 6 (CD44v6) exhibits a change in its expression pattern progressively from non-neoplastic, pre-malignant, and malignant (SCC) oral epithelial lesions. Cases with early features of invasion showed distinctly downregulated expression of CD44v6 protein whereas benign epithelial lesions expressed positive staining patterns comparable to those of the normal counterparts. Another marker for early oral cancer are the antigens recognized by monoclonal antibodies (MAbs) 17.13 and 63.12. These antibodies exhibit characteristic reactivity patterns in normal oral epithelium. Altered reactivity patterns of MAb 17.13 are associated with epithelial dysplasia and may be of assistance in detecting precancerous changes. The level of glutathione S-transferase (GST) activity has also been shown to correlate with the severity of oral epithelial dysplasia. Another method involves proliferation markers such as the centromere-associated protein CENP-F, which is a marker for cellular proliferation. In the basal and superficial cells of pre-malignant lesions, CENP-F has been shown to be increased compared to specimens from normal oral mucosa.

Silver cellular staining techniques have been used to quantitatively detect nucleolar organizer regions (AgNOR) in squamous cell nuclei in oral lesions. The quantitative features of AgNOR expression can discriminate between normal epithelium, dysplasia and carcinoma.

Further, while a strong correlation between DNA ploidy and oncogeneis has been demonstrated, a DNA ploidy analysis based on DNA concentration measurements through flow cytometry is subject to error. Detectable nuclear DNA content detectable may be altered due to the cellular mechanisms of replication, polyploidization, radiation therapy or vitamin B12 deficiency.

In short, considerable research effort has already been expended in characteristizing molecular diagnostic features as well as DNA ploidy of normal, dysplastic and malignant epithelial cells of the oral mucosa. Nonetheless, the practical task of assessing a patient's risk for developing oral squamous cell cancer still remains limited to the analysis of histopathologic features of standard microscopic preparations.

### SUMMARY OF THE INVENTION

It is an object of this invention to provide a system and method to detect epithelial dysplasia which utilizes non-lacerational trans-epithelial biopsy specimens based on combining computer-assisted cytological analysis and/or molecular diagnostic techniques for the purpose of increasing the sensitivity for detecting pre-cancerous and cancerous changes of the oral mucosa.

Both '218 and '219 describe a system for selecting cells using computer assisted analysis. This application describes the further use of molecular diagnostic techniques in the detection of dysplasia as well as further enhancing the system by conducting DNA ploidy analysis. By the inventors' knowledge, there are no systems which select suspect cells from a population in order to further assess such cells for atypical DNA ploidy.

An object of the present invention is to provide a pathologist with the means to retrieve the images of the epithelial cells which have been classified as atypical for a specific determination of DNA ploidy and/or further molecular diagnostic analysis.

Other objects, advantages and features of the invention will become more apparent hereinafter.

### BRIEF DESCRIPTION OF THE DRAWINGS

Diagram 1 is a flowchart of a method in accordance with one embodiment of the present invention which utilizes biomarker in the process of identifying cancerous and precancerous cells.

Diagram 2 is a flowchart of a method in accordance with another embodiment of the invention which utilizes a DNA ploidy analysis in the process of identifying cancerous and precancerous cells.

Diagram 3 is a flowchart of a method in accordance with the preferred embodiment of the invention which utilizes both biomarkers and a DNA ploidy analysis in the process of identifying cancerous and precancerous cells.

### DETAILED DESCRIPTION OF THE INVENTION AND THE PREFERRED EMBODIMENT

In the preferred embodiment, the presence of abnormal cellular morphology, abnormal keratinization and/or abnormal DNA ploidy, as detected by obtaining a non-lacerational trans-epithelial cellular sample, are combined with methods that demonstrate molecular alterations of cells from that trans-epithelial cellular sample to increase the sensitivity of 1) detection of epithelial lesions that are dysplastic or cancerous and 2) detection of epithelial lesions that will progress to carcinoma. An advantage of the subject invention over the prior art is greater sensitivity as an indicator of dysplasia and of a developing carcinoma, even preceding morphological tissue alterations. The trans-epithelial sample is preferably obtained using the device disclosed in the '186 application, the disclosure of which is fully incorporated herein by reference.

Epithelial lesions that display “atypical” cellular changes in a trans-epithelial cellular sample may or may not be of significance since some lesions represent carcinoma, others represent premalignancy and yet others represent benign lesions which may ultimately become malignant. By combining a DNA ploidy analysis of the trans-epithelial cellular specimen with a molecular diagnostics determination, the present invention can be utilized as a method of increasing the sensitivity for identifying those atypical epithelial lesions which will progress to carcinoma as well as identifying those which will not.

As an important feature of this invention, the trans-epithelial sample of epithelial tissue is examined for abnormalities in cellular morphology, DNA concentration, and keratinization as disclosed in the pending '218 and '219 applications and/or examined for other abnormalities in cellular morphology using computer assistance as disclosed in those applications. Atypical cells are selected for by the computer and a DNA ploidy determination of the suspect cells is then conducted by a pathologist.

Additionally, the sample may be analyzed with molecular diagnostic techniques including, but not limited to, fluorescence and non-fluroescence in situ hybridization, loss of heterozygosity, clonal genetic alterations, PCR, p53 expression and the expression pattern of CD44 variant 6 protein by immunohistochemistry, monoclonal antibodies reactivity patterns, glutathione S-transferase activity, quantitative assessment of nucleolar organizer regions and cell cycle and proliferation markers such as the centromere-associated protein.

Molecular diagnostic as well as DNA ploidy determination techniques that have been utilized to date have been performed on cellular specimens obtained from either invasive, lacerational biopsies or from scrapings of superficial cells using cytologic instruments. An advantage of this invention is the application of a DNA ploidy analysis and molecular diagnostic techniques to cellular samples obtained with a noninvasive apparatus such as that disclosed in the U.S. Pat. No. 6,258,044, which samples cells from all levels of an epithelial lesion. Another advantage of this invention is the increased sensitivity compared to all existing methods by themselves, including histopathology, cytology, and molecular diagnostic techniques of identifying dysplasia in epithelial tissue and the detection of epithelial lesions that may progress to carcinoma as well as those which may not.

The molecular diagnostic techniques can be applied before or after the trans-epithelial sample of epithelial tissue is examined for abnormalities in cellular morphology, abnormalities in keratinization or abnormalities in DNA ploidy as disclosed in the pending '218 and '219 applications and/or examined for other abnormalities in cellular morphology using computer assistance as disclosed in those applications. Furthermore, the DNA ploidy determination may be made either independently or in conjunction with the molecular diagnosis, but such DNA ploidy examination is always made in conjunction with the methods and systems of the '218 and '219 applications.

Because most of the interpretations of DNA measurements are population-based, the results of the computer analysis may be displayed as a DNA histogram. In a further embodiment, a histogram is plotted based on the DNA ploidy of the cell population. “Clean” cells, exhibiting normal nuclear to cytoplasmic ratios and morphology, are chosen from the population. This allows for the indication of atypical cells relative to the “normal” looking cells found within the same population and serves to eliminate the reduced sensitivity associated with using a blind control. Additionally, errors associated with estimating the DNA ploidy of a cell population are eliminated due to the fact that the final DNA ploidy determination is conducted by a pathologist on a cell by cell basis.

Dysplasia is characterized as being either high-risk (aneuploid), intermediate-risk (tetraploid) or a low risk (diploid) lesion. As the pathologist reviews the sample, an indicator on the histogram serves to represent the relative DNA ploidy determination found for an individual cell of interest. In a preferred embodiment, a light indicator on the histogram alerts the pathologist as to the DNA ploidy of the selected cell of interest.

### Results

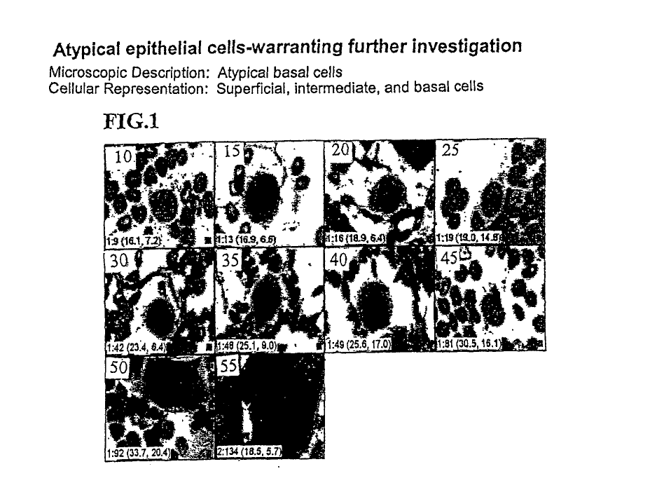

FIGS. 1-3 present data from superficial, intermediate and basal cell layers of the oral cavity. Each quadrant contains a suspect cell found within the population under review and includes a nuclear to cytoplasmic ratio displayed in the bottom left hand corner.

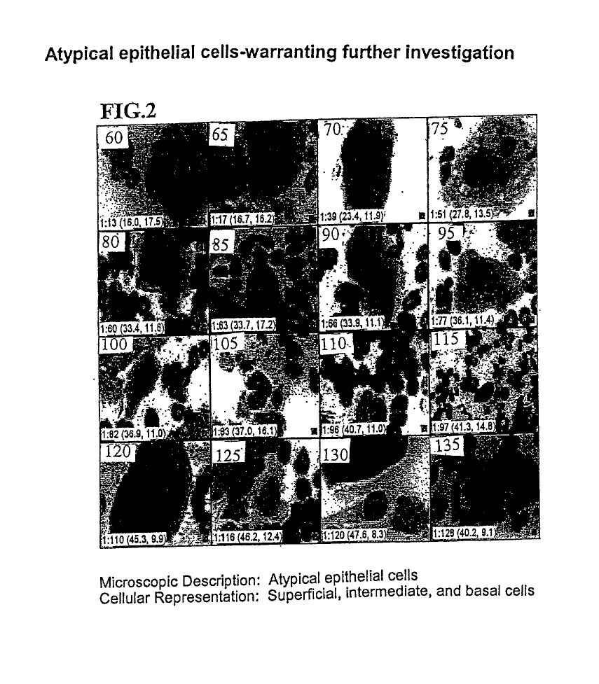

FIGS. 1 and 2 display atypical cells warranting further investigation of the respective patient. Both Figs. show an increase in the nuclear staining, an increase in the nuclear cytoplasmic ratio, and nuclear crowding with a loss of polarity.

In FIG. 1, quadrants 10, 15, 20, 25, 30, 35, 40, 45, 50, and 55 show an increase in nuclear staining. Of special concern, quadrant 10 indicates that the cell of interest has a high nuclear to cytoplasmic ratio (of 1:9). This is observed by an increase in density as the nucleus absorbs a larger portion of the cytometric dye. The ability to examine individual cells of interest gives the pathologist a greater degree of accuracy. Further investigation may include additional harvesting of cells from the region of interest.

Similarly, FIG. 2 displays cells of a second patient also warranting further investigation by a pathologist. Quadrants 60 and 65 indicate a relatively high nuclear to cytoplasmic ratio of 1 to 13 and 1 to 17, respectively. Of additional concern, quadrants 125 and 130 contain naked nuclei surrounded by a bloody background. By examining the actual cell the pathologist is able to determine that the low nuclear to cytoplasmic ratio is attributed to a cell which is no longer intact.

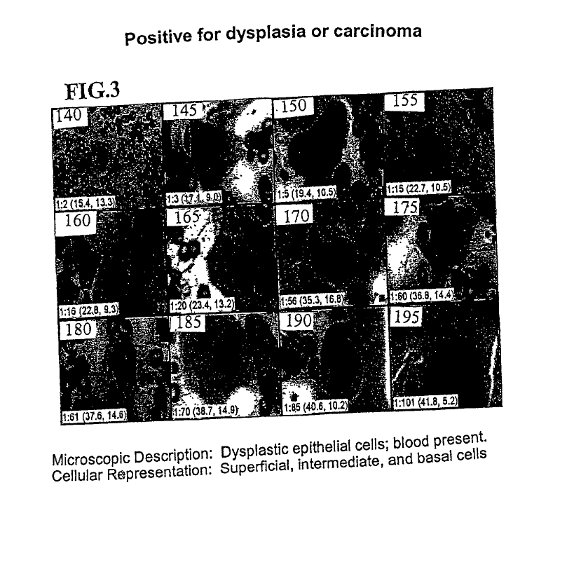

FIG. 3. shows cells positive for dysplasia or carcinoma. As indicated by the display in the bottom left hand corner of quadrants 150, 155 and 160, there is a dramatic increase in the nuclear to cytoplasmic ratio. Upon further observance by a pathologist, it is noted the cells have an irregular shape. The computer based retrieval of cells containing a combination of irregular shape and nuclear DNA concentration allows the pathologist to quickly focus on cells of interest. Again, regions of interest may be revisited and additional cells harvested by the pathologist.

The final interpretation of the image analysis histogram may be conducted in conjunction with the patient's history, biopsy findings, or any other pertinent test results. For example: all the image results may then be integrated into the corresponding biopsy report and discrepancies between the two addressed.

Having described this invention with regard to specific embodiments, it is to be understood that the description is not meant as a limitation since further embodiments, modifications and variations may be apparent or may suggest themselves to those skilled in the art. It is intended that the present application cover all such embodiments, modifications and variations.

Diagram 1
Taking a trans-epithelial sample of epithelial tissue
↓
Examining said trans-epithelial sample of epithelial tissue
for abnormalities in cellular morphology and abnormalities
in keratinization and/or examining said sample of epithelial
tissue for abnormalities using computer-assisted analysis,
including but not limited to the machines and/or
techniques of the ‘218 and/or ‘219 applications.
↓
Analyzing the sample with a molecular diagnostic technique,
said technique including but not limited to, fluorescence
in situ hybridization, loss of heterozygosity, clonal genetic
alterations, PCR, p53 expression and the expression pattern
of CD44 variant 6 protein by immunohistochemistry,
monoclonal antibodies reactivity patterns, glutathione
S-transferase activity, measuring the number of nucleolar
organizer regions and cell-cycle and proliferation markers such
as the centromere-associated protein.

Diagram 2
Taking a trans-epithelial sample of epithelial tissue
↓
Examining said trans-epithelial sample of epithelial tissue
for abnormalities in cellular morphology, and DNA concentration
and/or examining said sample of epithelial
tissue for abnormalities using computer-assisted analysis,
including but not limited to the machines and/or
techniques of the ‘218 and/or ‘219 applications.
↓
Analyzing the sample for a DNA ploidy analysis, said DNA ploidy
determination being conducted by a pathologist.

Diagram 3
Taking a trans-epithelial sample of epithelial tissue
↓
Examining said trans-epithelial sample of epithelial tissue
for abnormalities in cellular morphology, keratinization and DNA
concentration and/or examining said sample of epithelial
tissue for abnormalities using computer-assisted analysis,
including but not limited to the machines and/or
techniques of the ‘218 and/or ‘219 applications.
↓
Analyzing the sample with a molecular diagnostic technique and/or for
a DNA ploidy analysis, said DNA ploidy determination being conducted
by a pathologist and said molecular diagnostic technique including but not
limited to, fluorescence in situ hybridization, loss of heterozygosity,
clonal genetic alterations, PCR, p53 expression and the expression pattern
of CD44 variant 6 protein by immunohistochemistry,
monoclonal antibodies reactivity patterns, glutathione
S-transferase activity, measuring the number of nucleolar
organizer regions and cell-cycle and proliferation markers such
as the centromere-associated protein.

What is claimed is:

1. A method for detecting epithelial dysplasia comprising the steps of: taking a non-lacerational trans-epithelial sample of epithelial tissue and then analyzing the sample with a molecular diagnostic technique.

2. A method as claimed in claim 1, wherein said technique includes, but is not limited to, fluorescence and non-fluorescence in situ hybridization, loss of heterozygosity, clonal genetic alterations, PCR, p53 expression and the expression pattern of CD44 variant 6 protein by immunohistochemistry, monoclonal antibodies reactivity patterns, glutathione S-transferase activity, measuring the number of nucleolar organizer regions, and cell-cycle and proliferation markers such as the centromere-associated protein.

3. A method as claimed in claim 1, wherein said trans-epithelial sample of epithelial tissue is examined for abnormalities in cellular morphology and abnormalities in keratinization.

4. A method as claimed in claim 1, wherein said trans-epithelial sample of epithelial tissue is examined for abnormalities using computer-assisted analysis.

5. A method for detecting epithelial dysplasia comprising the steps of:
(a) examining said trans-epithelial sample of epithelial tissue for abnormalities in cellular morphology and abnormalities in keratinization;
(b) and then analyzing the sample with a molecular diagnostic technique.

6. A method as claimed in claim 5, wherein said technique includes, but is not limited to, fluorescence or non-flurescence in situ hybridization, loss of heterozygosity, clonal genetic alterations, PCR, p53 expression and the expression pattern of CD44 variant 6 protein by immunohistochemistry, monoclonal antibodies reactivity patterns, glutathione S-transferase activity, measuring the number of nucleolar organizer regions and cell-cycle and proliferation markers such as the centromere-associated protein.

7. A method as claimed in claim 5, wherein said trans-epithelial sample of epithelial tissue is examined for abnormalities using computer-assisted analysis.

8. A method for detecting precancerous and cancerous cells comprising:
(a) analyzing a population of cells to determine the most suspect cells therein; and
(b) conduction a ploidy analysis on said most suspect cells on a cell by cell basis.

9. The method of claim 8, wherein said analyzing said population of cells is conducted by computer analysis.

10. The method of claim 9, wherein said population of cells are harvested from tissue from a human.

11. The method of claim 10, wherein said population of cells are harvested from epithelial tissue.

12. The method of claim 11, wherein said population of cells are harvested from the superficial, intermediate and basal cell layers.

13. The method of claim 12, wherein said population of cells are obtained by use of a non-lacerational biopsy.

14. The system of claim 8, wherein said atypical cells are further distinguished with a molecular diagnostic technique.

15. A method of enhancing cancerous cell diagnosis of a population of cells which have been identified as having suspect cancerous cells, said method comprising conducting ploidy analysis on said suspect cancerous cells on a cell by cell basis.

16. A system to detect cancerous and precancerous cells in a cell population comprising:
(a) a computer to morphologically analyze individual cells for atypicality;
(b) said computer cytometrically analyzing said individual cells for atypicality; and
(c) selecting said individual cells exhibiting atypical cytometry and morphology in order to conduct DNA ploidy quantization.

17. The system of claim 16, wherein said cancerous and precancerous cells are examined for abnormalities using computer assisted analysis.

18. The system of claim 17, wherein the location of said cells exhibiting both atypical morphology and cytometry are retrieved by said computer for DNA ploidy analysis by a pathologist on a cell by cell basis.

19. The system of claim 18, wherein a histogram is plotted based of the DNA ploidy of said cell population.

20. The system of claim 18, wherein said atypical cells are selected based on reference cells chosen from the same population.

21. The system of claim 19, wherein said histogram has a light indicator to further indicate the DNA ploidy of said atypical cell during final analysis by a pathologist.

22. The system of claim 21, wherein the final interpretation of the image analysis histogram is conducted in conjunction with the patient's history, biopsy findings, or any other pertinent test results.

23. The system of claim 16, comprising the further distinction of said cells with molecular diagnostic techniques.
